(19) United States
(12) Patent Application Publication
(10) Pub. No.: US 2026/0249040 A1
(43) Pub. Date: Aug. 27, 2026

(54) SLEEP-AID AUDIO DEVICE

(71) Applicant: Shenzhen DreamCove Tech Co.,Ltd, Shenzhen (CN)

(72) Inventor: Jianping Wang, Shenzhen (CN)

(21) Appl. No.: 19/648,075

(22) Filed: Apr. 15, 2026

(30) Foreign Application Priority Data
Mar. 4, 2026 (CN) 202610256747.6

(51) Int. Cl. A61M 21/02; A61M 21/00

(52) U.S. Cl. CPC A61M 21/02; A61M 2021/0027; A61M 2205/43; A61M 2205/8206

(57) ABSTRACT

A sleep-aid audio device is provided, including: a main housing. A control panel assembly is mounted at a top of the main housing. One side of the main housing is connected to a wristband assembly through a spring pin. A wristband buckle is mounted at a top of the wristband assembly. An internal supporting structure is fixedly connected to a lighting assembly, a sound production assembly, a control circuit board, and a battery assembly respectively. Independent positioning and stable assembling of core assemblies inside the main housing of the sleep-aid audio device effectively avoids a problem of assembly displacement caused by equipment vibration and ensures continuity of sound outputting and stability of lighting presentation. A speaker of the sound production assembly is mounted within a closed sound chamber enclosed by a sound chamber shell and an internal supporting structure, and a sound production end is also fitted with a diaphragm.

### TECHNICAL FIELD

The present disclosure belongs to the technical field of speakers, and specifically relates to a sleep-aid audio device.

### BACKGROUND

With the continuously increasing demand of residents for sleep health, sleep-aid audio devices that are integrated with sound soothing and lighting assistance functions have become mainstream smart home devices. After searching, a sleep-aid audio device No. "CN217656719U" in the existing technology achieves sleep assistance by cooperation between a sound effect and lighting by integration of a sound production device and ambient lights. However, an internal structural layout design of the sleep-aid audio device has significant irrationality. This technology only uses a protective chamber box to simply store core assemblies such as a sound production assembly and a lighting assembly, without a targeted layered assembling design. It does not have an independent sound chamber shell, lampshade, and battery compartment structure, and a supporting structure is only a simple fixed bracket. Assemblies such as a speaker, a control circuit board, and a battery do not have dedicated brackets for precise positioning. During the use of the equipment, an assembly moves easily due to slight vibrations. This not only causes noises and sound interruption in sound effects outputted by the speaker, but also causes a light emission angle of the lighting assembly to shift, so that the sleep-aid synergy effect of the lighting and the sound effect is greatly reduced. Meanwhile, a messy internal structure also reduces the assembling efficiency of the product and increases the difficulty of later maintenance.

Moreover, there are still many shortcomings in the compatibility between the design of a sleep-aid function of the existing sleep-aid audio device and an actual sleeping need of a user. The lighting assembly of the above type of sleep-aid audio device only includes an ordinary light-emitting diode and a basic lampshade that match each other. The lampshade has no optimized design of a light guiding structure, so that light directly diverges outward. Thus, non-uniform light distribution and extremely high local brightness easily occur. Dazzling light cannot achieve a sleep-aid effect of relaxing the nerves, but affects sleeping of the user. With no closed sound chamber design, the sound production assembly has a low reproduction degree in simulating a natural sleep-aid sound effect and a poor low-frequency sound effect, lacks a sense of depth and immersion in the sound effect, and has a poor soothing effect. Meanwhile, a key assembly of this type of product has a single function and insufficient indicator light configuration, and cannot achieve fine operations such as sleep-aid sound effect switching, lighting mode adjustment, and timing duration setting. User operations are cumbersome and cannot respond to sleep-aid operation needs in a timely manner. In addition, most products do not have a dedicated battery compartment design, so that the space is used improperly, causing poor portability of the equipment. This product also lacks an exterior decoration structure and a charging interface protection design. This further reduces an overall user experience of the product.

### SUMMARY

The present disclosure aims to provide a sleep-aid audio device, and aims to solve the problems in the background section.

To achieve the above objectives, the present disclosure provides the following technical solution: A sleep-aid audio device includes a main housing. A control panel assembly is mounted at a top of the main housing. One side of the main housing is connected to a wristband assembly through a spring pin. A wristband buckle is mounted at a top of the wristband assembly.

A sound chamber shell, a lampshade, an inner shell, and a battery compartment are sequentially mounted inside the main housing from bottom to top. A lighting assembly is mounted on an inner side of the lampshade. A sound production assembly is mounted on an inner side of the sound chamber shell. A control circuit board is arranged at a top of the battery compartment. A battery assembly is mounted inside the battery compartment. An internal supporting structure is further disposed within the main housing. The internal supporting structure is fixedly connected to the lighting assembly, the sound production assembly, the control circuit board, and the battery assembly respectively. A decorative ring is mounted at the top of the main housing in an embedded manner.

In a preferred technical solution of the present disclosure, the control panel assembly includes a control panel. The control panel is mounted at an inner top end of the main housing in an abutting manner. A plurality of functional keys that penetrate through the top of the main housing are mounted on a surface of the control panel. The functional keys are electrically connected to the control circuit board. The functional keys include a power key, a volume increase/decrease key, a mode switching key, a timer key, a lighting key, a child lock key, a Bluetooth key, and a sound effect selection key. At least three indicator lights penetrating through the top of the main housing are further embedded into the surface of the control panel. The indicator lights are electrically connected to the control circuit board.

In a preferred technical solution of the present disclosure, an inner wall of the lampshade is coated with a light guiding coating, and the lampshade is sleeved inside the sound chamber shell.

In a preferred technical solution of the present disclosure, the wristband assembly includes a wristband body and the wristband buckle. A buckle hole is formed in a surface of the wristband body. The wristband body is wound end-to-end and is fixed through the wristband buckle passing through the buckle hole, to form an annular band. One end of the wristband body forming the annular band is internally inserted with the spring pin. Two ends of the spring pin are inserted into open slots formed in an outer side of the inner shell.

In a preferred technical solution of the present disclosure, the lighting assembly includes a light-emitting diode (LED) board. The LED board is of an annular plate-like structure. The LED board is fixed to a top of the lampshade in an abutting manner. A plurality of LEDs are arrayed on a surface of the LED board. The lampshade is made of high-transparency frosted acrylic material. The lampshade is covered outside the LEDs.

In a preferred technical solution of the present disclosure, the sound production assembly includes a speaker and a diaphragm. The speaker is fixedly mounted in a sound chamber enclosed by the sound chamber shell and the internal supporting structure. The sound chamber is of a closed chamber structure. The diaphragm is mounted at a sound production end of the speaker in an abutting manner. The sound chamber shell is made of a transparent plastic material.

In a preferred technical solution of the present disclosure, the control circuit board includes a main control chip and a storage module. The main control chip is electrically connected to the functional keys, the lighting assembly, the sound production assembly, and the battery assembly respectively. The storage module is electrically connected to the main control chip. Sleep-aid audios are pre-stored in the storage module. The main control chip is also integrated with a Bluetooth chip.

In a preferred technical solution of the present disclosure, the battery assembly includes a rechargeable battery and a charging interface. The rechargeable battery is clamped and fixed inside the battery compartment. The rechargeable battery is electrically connected to the control circuit board. The charging interface is embedded in an outer charging slot of the main housing. The charging interface is electrically connected to the rechargeable battery.

In a preferred technical solution of the present disclosure, the internal supporting structure includes a speaker bracket, a circuit board bracket, and a battery compartment bracket. The speaker bracket is clamped and fixed to an inner wall of the inner shell. An interior of the speaker bracket is fixedly connected to the speaker. The circuit board bracket is integrally formed at a top of the inner shell. The circuit board bracket is configured to screw and fix the control circuit board. The battery compartment bracket encloses a side wall of the battery compartment. The battery compartment bracket is configured to clamp and fix the rechargeable battery.

In a preferred technical solution of the present disclosure, the decorative ring is made of a metal material or a frosted plastic material, and the decorative ring surrounds and is arranged outside the functional keys of the control panel assembly.

Compared with the prior art, the present disclosure has the beneficial effects below:

1) The sound chamber shell, the lampshade, the inner shell, and the battery compartment are sequentially mounted inside the main housing of the sleep-aid audio device of the present disclosure from bottom to top, and the speaker bracket, the circuit board bracket, and the compartment bracket of the internal supporting structure are precisely and fixedly connected to the sound production assembly, the control circuit board, and the battery assembly respectively, thus achieving independent positioning and stable assembling of the core assemblies, effectively avoiding a problem of assembly displacement caused by equipment vibration and ensuring continuity of sound outputting and stability of lighting presentation. Meanwhile, a standardized layered structure also greatly enhances the assembling efficiency of a product. Furthermore, a speaker of the sound production assembly is mounted within the closed sound chamber enclosed by the sound chamber shell and the internal supporting structure, and the sound production end is also fitted with the diaphragm. Low-frequency performance and three-dimensional immersion of a sleep-aid sound effect are greatly enhanced. A reproduction degree of a natural sleep-aid sound effect such as child sounds and wind and wave sounds is higher, and the sleep-aid and soothing effects of sound effects are significantly enhanced.

2) In the lighting assembly of the present disclosure, the annular plate-like LED board is fixed to the top of the lampshade in the abutting manner. Due to the light guiding coating on the inner wall of the lampshade and the high-transparency frosted acrylic material, light emitted by the LEDs can be led out uniformly after diffuse reflection. The light is soft and not dazzling, so that a perfect nerve-soothing sleep-aid lighting environment is created. The control panel assembly is integrated with various functional keys such as the power key, the volume increase/decrease key, the sound effect selection key, and the lighting key, and is provided with the at least three indicator lights. Furthermore, the keys and the indicator lights are electrically connected to the control circuit board, so that a user can quickly complete operations such as switching sleep-aid sound effects, adjusting a lighting mode, and setting timing duration. This enhances timeliness and convenience of the sleep-aid operation. Meanwhile, the main control chip of the control circuit board is integrated with the Bluetooth chip. The storage module is pre-stored with the sleep-aid audios. This implements wireless sound effect expansion and enriches options of sleep-aid sound effects. The battery compartment can be clamped and fixed to the rechargeable battery. The charging interface is embedded into the outer charging slot of the main housing, so that it optimizes the internal space, enhances the convenience of the equipment, and achieves effective protection on the charging interface. The decorative ring embedded into the top of the main housing surrounds outside the functional keys, so that the appearance texture of the product is further enhanced. The detachable design of the wristband assembly balances wearing of the equipment and a demand for placement and use, thus effectively optimizing a comprehensive user experience of the product.

### BRIEF DESCRIPTION OF THE DRAWINGS

The accompanying drawings are intended to provide a further understanding of the present disclosure and constitute a part of this specification. The accompanying drawings and embodiments of the present disclosure are used together to explain the present disclosure rather than constituting a limitation on the present disclosure. In the accompanying drawings:

FIG. 1 is a schematic diagram of a structure of the present disclosure;

FIG. 2 is a first schematic diagram of a structure of the present disclosure;

FIG. 3 is a cross-sectional view of the present disclosure;

FIG. 4 is a schematic exploded diagram of the present disclosure;

FIG. 5 is a first schematic exploded diagram of the present disclosure;

FIG. 6 is a schematic diagram of a structure of a control panel assembly of the present disclosure; and

FIG. 7 is a schematic diagram of a structure of an inner shell of the present disclosure.

In the drawings: 100: main housing; 101: sound chamber shell; 102: lampshade; 103: inner shell; 1031: open slot; 104: battery compartment; 110: control panel assembly; 111: control panel; 112: functional key; 1121: power key; 1122: volume increase/decrease key; 1123: mode switching key; 1124: timer key; 1125: lighting key; 1126: child lock key; 1127: sound effect selection key; 113: indicator light; 114: light guiding coating; 120: decorative ring; 200: wristband assembly; 201: wristband body; 210: spring pin; 220: wristband buckle; 300: lighting assembly; 301: LED board; 302: LED; 400: sound production assembly; 401: speaker; 402: sound chamber; 403: diaphragm; 500: control circuit board; 501: main control chip; 502: storage module; 600: battery assembly; 601: rechargeable battery; 602: charging interface; 700: internal supporting structure; 701: speaker bracket; 702: circuit board bracket; and 703: battery compartment bracket.

### DETAILED DESCRIPTION OF THE EMBODIMENTS

The following clearly and completely describes the technical solutions in the embodiments of the present disclosure with reference to the accompanying drawings in the embodiments of the present disclosure. Apparently, the described embodiments are some of the embodiments of the present disclosure rather than all the embodiments. All other embodiments obtained by a person of ordinary skill in the art based on the disclosed embodiments without creative efforts shall fall within the protection scope of the present disclosure.

### Embodiment

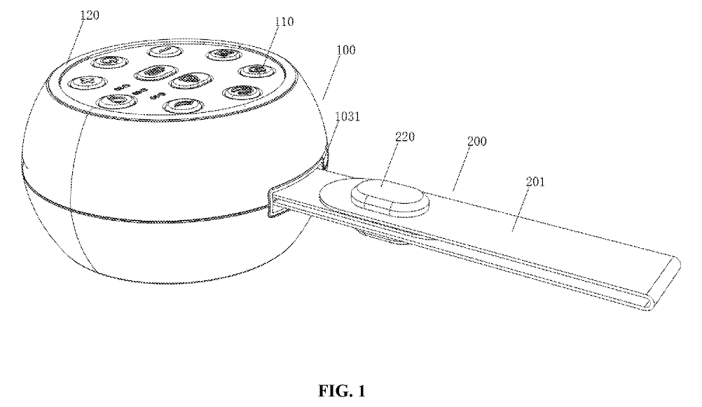

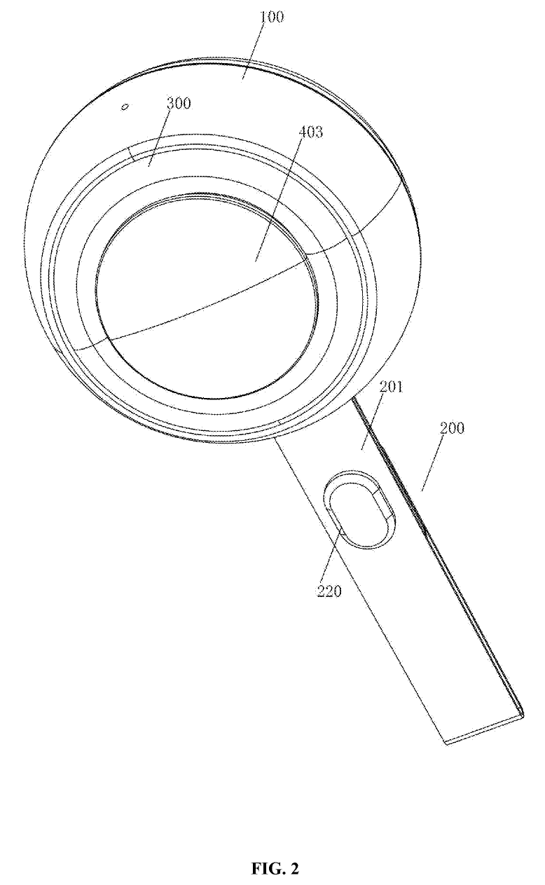

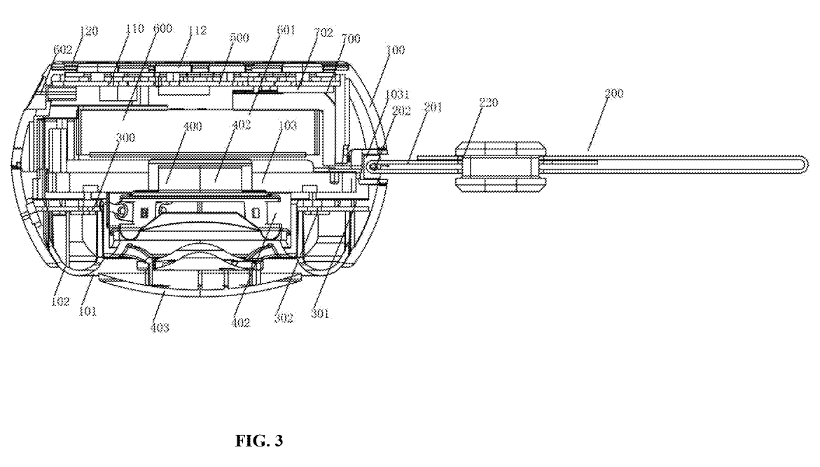

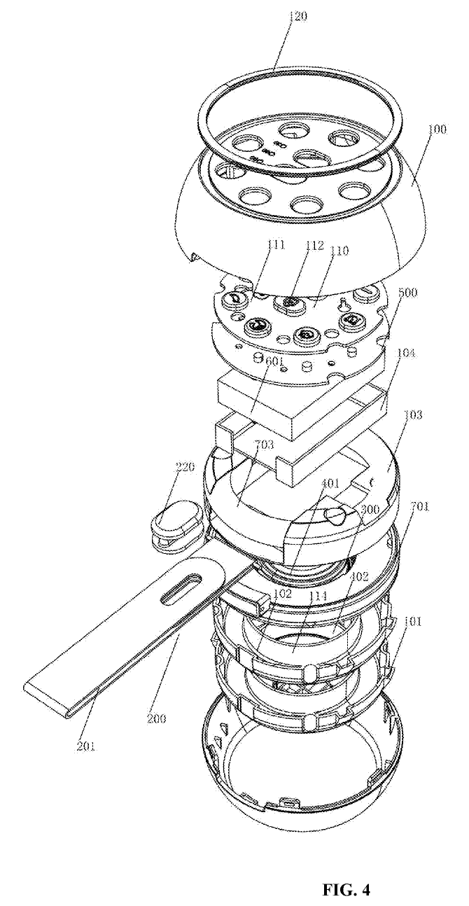

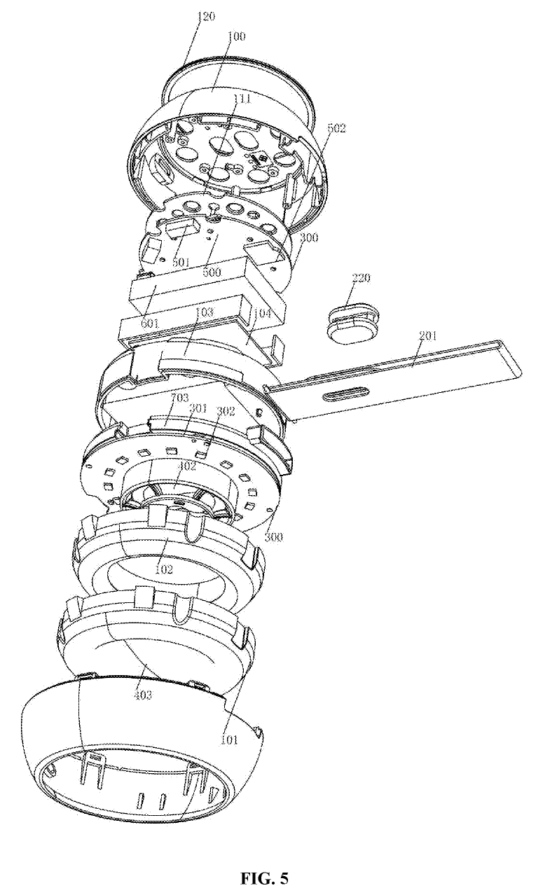

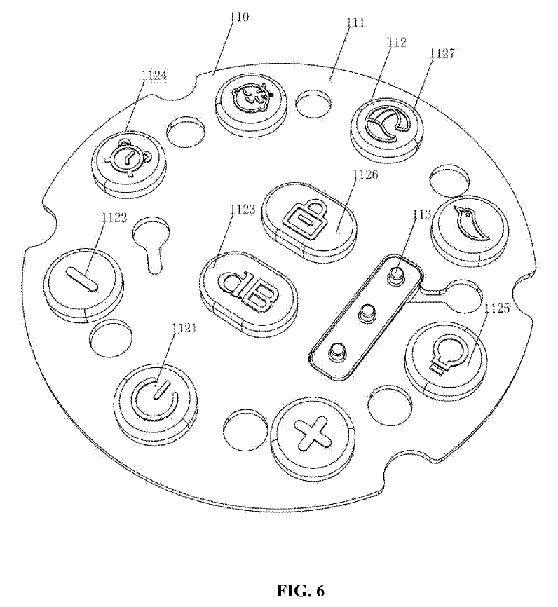

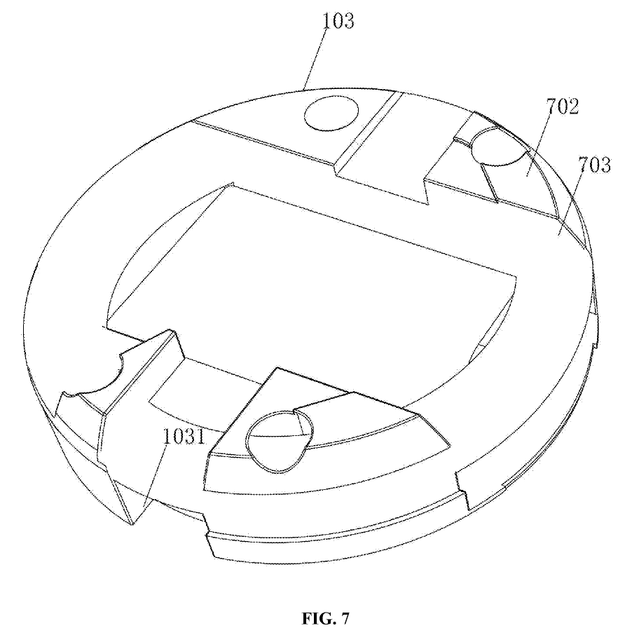

Referring to FIG. 1 to FIG. 7, the present disclosure provides the following technical solution: A sleep-aid audio device includes a main housing 100. A control panel assembly 110 is mounted at a top of the main housing 100. One side of the main housing 100 is connected to a wristband assembly 200 through a spring pin 210. A wristband buckle 220 is mounted at a top of the wristband assembly 200.

A sound chamber shell 101, a lampshade 102, an inner shell 103, and a battery compartment 104 are sequentially mounted inside the main housing 100 from bottom to top. A lighting assembly 300 is mounted on an inner side of the lampshade 102. A sound production assembly 400 is mounted on an inner side of the sound chamber shell 101. A control circuit board 500 is arranged at a top of the battery compartment 104. A battery assembly 600 is mounted inside the battery compartment 104. An internal supporting structure 700 is further disposed within the main housing 100. The internal supporting structure 700 is fixedly connected to the lighting assembly 300, the sound production assembly 400, the control circuit board 500, and the battery assembly 600 respectively.

A decorative ring 120 is mounted at the top of the main housing 100 in an embedded manner.

Specifically, when the sleep-aid audio device works, the control panel assembly 110 is located at the top of the main housing 100 and can achieve user operation interactions. The wristband assembly 200 is movably connected to the main housing 100 through the spring pin 210, so that the wristband buckle 220 can achieve quick mounting and removal of the wristband assembly 200, to meet a demand for switching between portable carrying and placement and use

Layered assembled structures from bottom to top are used inside the main housing 100. The sound chamber shell 101, the lampshade 102, the inner shell 103, and the battery compartment 104 are sequentially positioned and mounted. The lighting assembly 300 achieves soft lighting outputting on the inner side of the lampshade 102. The sound production assembly 400 can play back sleep-aid sound effects on the inner side of the sound chamber shell 101. The control circuit board 500 and the battery assembly 600 respectively implement control and power supplying at the top of and inside the battery compartment 104. The internal supporting structure 700 provided is fixedly connected to the lighting assembly 300, the sound production assembly 400, the control circuit board 500, and the battery assembly 600 respectively, thus avoiding assembly displacement caused by vibration during use of the equipment and ensuring stable outputting of light and sound effects. The decorative ring 120 at the top of the main housing 100 enhances the appearance texture and also plays a role in protecting the assembly at the top, to enhance the quality of the product.

In this embodiment, the control panel assembly 110 includes a control panel 111. The control panel 111 is mounted at an inner top end of the main housing 100 in an abutting manner. A plurality of functional keys 112 that penetrate through the top of the main housing 100 are mounted on a surface of the control panel 111. The functional keys 112 are electrically connected to the control circuit board 500. The functional keys 112 include a power key 1121, a volume increase/decrease key 1122, a mode switching key 1123, a timer key 1124, a lighting key 1125, a child lock key 1126, a Bluetooth key, and a sound effect selection key 1127. At least three indicator lights 113 penetrating through the top of the main housing 100 are further embedded into the surface of the control panel 111. The indicator lights 113 are electrically connected to the control circuit board 500.

Specifically, the functional keys 112 provided penetrate through the top of the main housing 100 and extend to the outside, making it convenient for a user to directly touch and operate the functional keys 112. The functional keys 112 are electrically connected to the control circuit board 500. The power key 1121 is pressed to start and stop the equipment. The volume increase/decrease key 1122 adjusts the volume of a sound effect. The mode switching key 1123 switches a working mode. The timer key 1124 sets sleep-aid duration. The lighting key 1125 adjusts a lighting state. The child lock key 1126 avoids accidental touch. The Bluetooth key enables wireless connection. The sound effect selection key 1127 switches sleep-aid audios such as child sounds and wind and wave sounds. All key instructions are transmitted in real time to the control circuit board 500 to complete instruction parsing.

The indicator lights 113 also penetrate through the top of the main housing 100 and is electrically connected to the control circuit board 500. The at least three indicator lights 113 can correspond to different timing durations or working states, and provide intuitive feedback on operation conditions of the equipment through on/off changes. This allows a user to quickly control the state of the equipment without complex operations, thus achieving convenient and visual control of a sleep-aid function.

In this embodiment, an inner wall of the lampshade 102 is coated with a light guiding coating 114, and the lampshade 102 is sleeved inside the sound chamber shell 101.

Specifically, when the lampshade 102 works in conjunction with the lighting assembly 300, the lampshade 102 is sleeved inside the sound chamber shell 101. This achieves precise positioning with an internal structure and provides a closed and stable lighting environment for the lighting assembly 300. The light guiding coating 114 coated on the inner wall of the lampshade 102 can further comb and diffuse the light emitted by the lighting assembly 300, to avoid dazzling caused by direct lighting.

The light guiding coating 114 can convert a point light source of the LEDs 302 into a uniform surface light source, thus making the light softer and gentler and adapting to a lighting atmosphere required for sleep aid. The sleeved structure between the lampshade 102 and the sound chamber shell 101 ensures stability of the mounting of the lighting assembly 300 and avoids a waste caused by leakage of internal light. Meanwhile, it does not affect the sound effect propagation of the sound production assembly 400, so that sleep-aid functions of lighting soothing and sound relaxing work together. The cooperation between the light guiding coating 114 and the lampshade 102 optimizes a light outputting effect structurally, solves problems that a traditional sleep-aid audio device has dazzling light and non-uniform light distribution, and further enhances the comfort of sleep-aid lighting.

In this embodiment, the wristband assembly 200 includes a wristband body 201 and the wristband buckle 220. A buckle hole is formed in a surface of the wristband body 201. The wristband body 201 is wound end-to-end and is fixed through the wristband buckle 220 passing through the buckle hole, to form an annular band. One end of the wristband body (201) forming the annular band is internally inserted with the spring pin 210. Two ends of the spring pin 210 are inserted into open slots 1031 formed in an outer side of the inner shell 103.

Specifically, when the audio device is in use, the wristband body 201 can be easily worn by a user. An annular wearing structure is formed by interweaving cooperation between the buckle hole in the surface and the wristband buckle 220, to meet a demand for user wrist wearing or a demand for equipment hanging. The spring pin 210 is inserted inside an end of the wristband body 201, and the two ends of the spring pin 210 are precisely inserted into the open slots 1031 on the outer side of the inner shell 103, thus achieving detachable connection between the wristband assembly 200 and the main shell 100. The insert fit manner can complete mounting and removal of the wristband assembly 200 without a tool, thus making it convenient for the user to quickly replace or remove the wristband based on a usage scenario. The open slots 1031 play a role in limiting and fixing the spring pin 210, to avoid the wristband assembly 200 from being loosened and falling off during wearing. While ensuring the convenience of removal, it enhances the flexibility of carrying of the equipment, so that normal work of the assemblies inside the main housing 100 is not affected, the sleep-aid audio device can be used at any time anywhere, and specific usage scenarios are expanded.

In this embodiment, the lighting assembly 300 includes an LED board 301. The LED board 301 is of an annular plate-like structure. The LED board 301 is fixed to a top of the lampshade 102 in an abutting manner. A plurality of LEDs 302 are arrayed on a surface of the LED board 301. The lampshade 102 is made of high-transparency frosted acrylic material. The lampshade 102 is covered outside the LEDs 302.

Specifically, when the lighting assembly 300 works, the LED board 301 that is of the annular plate-like structure is fixed to the top of the lampshade 102 in the abutting manner, thus ensuring that light emission angles of the LEDs 302 are uniform and stable. The plurality of LEDs 302 are arrayed on the surface of the LED board 301, and can emit soft sleep-aid light after being powered on. The lampshade 102 is made of the high-transparency frosted acrylic material and is covered outside the LEDs 302. This can ensure smooth light transmission and weaken strong light points of the LEDs through the frosted texture, thus avoiding direct light stimulation to eyes. The design of abutment between the annular LED board 301 and the lampshade 102 allows the light to spread uniformly along the lampshade 102, to form surrounding soft lighting that works together with a sleep-aid sound effect to create a quiet sleeping environment.

A fixing structure for the LED board 301 avoids light deviation caused by the displacement of the LEDs. The material and structure of the lampshade 102 cooperate to optimize the lighting effect in the entire process of light emission, light guiding, and light dispersion, so that the light always maintains a gentle and non-dazzling state, to continuously provide a user with a visual sleep-aid support.

In this embodiment, the sound production assembly 400 includes a speaker 401 and a diaphragm 403. The speaker 401 is fixedly mounted in a sound chamber 402 enclosed by the sound chamber shell 101 and the internal supporting structure 700. The sound chamber 402 is of a closed chamber structure. The diaphragm 403 is mounted at a sound production end of the speaker 401 in an abutting manner. The sound chamber shell 101 is made of a transparent plastic material.

Specifically, when the sound production assembly 400 works, the speaker 401 is fixed in the closed sound chamber 402 enclosed by the sound chamber shell 101 and the internal supporting structure 700. The closed chamber structure can effectively gather sound effects and avoid loss in sound quality due to sound leakage. Sounds produced by the speaker 401 after the speaker is powered on resonates in the sound chamber 402, thus enhancing the stereo perception and fullness of a sound effect. The diaphragm 403 is mounted at the sound production end of the speaker 401 in the abutting manner. This can enhance the low-frequency sound effect performance, to achieve a higher reproduction degree in sleep-aid audios such as child sounds, wind and wave sounds, and raining sounds, so that the sound effect is more natural, and the soothing effect is more outstanding.

The sound chamber shell 101 is made of the transparent plastic material. This does not affect the light transmission of the lighting assembly 300, and also provides a stable mounting foundation for the sound production assembly 400. The internal supporting structure 700 fixes the speaker 401 to avoid noises caused by displacement of the speaker 401 due to equipment vibrations, thus making sounds more immersive and soothing and accurately meeting an auditory sleep-aid need of a user.

In this embodiment, the control circuit board 500 includes a main control chip 501 and a storage module 502. The main control chip 501 is electrically connected to the functional keys 112, the lighting assembly 300, the sound production assembly 400, and the battery assembly 600 respectively. The storage module 502 is electrically connected to the main control chip 501. Sleep-aid audios are pre-stored in the storage module 502. The main control chip 501 is also integrated with a Bluetooth chip.

Specifically, the control circuit board 500 can receive operation instructions from the functional keys 112 in real time during working, and quickly parse the operation instructions to issue execution commands to the assemblies, thus achieving unified control of functions such as lighting adjustment, sound effect switching, and mode control.

The storage module 502 is electrically connected to the main control chip 501, and various sleep-aid audios are pre-stored inside the storage module 502. The main control chip 501 can directly invoke audio data in the storage module 502 and transmit the audio data to the sound production assembly 400 for playback, so that sleep-aid sound effect outputting can be achieved without external equipment. The Bluetooth chip integrated into the main control chip 501 can expand a wireless connection function, to support transmission of audios by external equipment, enrich sources of sleep-aid sound effects, and provide a core control support for the intelligent and diversified operations of sleep-aid functions.

In this embodiment, the battery assembly 600 includes a rechargeable battery 601 and a charging interface 602. The rechargeable battery 601 is clamped and fixed inside the battery compartment 104. The rechargeable battery 601 is electrically connected to the control circuit board 500. The charging interface 602 is embedded in an outer charging slot of the main housing 100. The charging interface 602 is electrically connected to the rechargeable battery 601.

Specifically, the rechargeable battery 601 is clamped and fixed inside the battery compartment 104. A wrapping structure of the battery compartment 104 prevents the rechargeable battery 601 from shaking and moving, thus ensuring stable power supplying. The rechargeable battery 601 is electrically connected to the control circuit board 500, to continuously provide stable power for all electrical assemblies such as the lighting assembly 300, the sound production assembly 400, and the control circuit board 500, to ensure long-term continuous operation of the equipment. The charging interface 602 is embedded into the outer charging slot of the main housing 100, which facilitates connection to a charging cable by a user to replenish the power of the rechargeable battery 601, and avoids direct exposure of the charging interface 602 through a protective structure of the charging slot, thus reducing entering of dust and water vapor to damage the interface and prolonging the service life of the equipment. The stable power supplying of the rechargeable battery 601 cooperates with the safe charging of the charging interface 602, so that the problem in the battery life for outdoor use of the sleep-aid audio device. A fixing structure of the battery compartment 104 further ensures the power supplying stability, so that the equipment can continuously and stably output a sleep-aid function in any scenario.

In this embodiment, the internal supporting structure 700 includes a speaker bracket 701, a circuit board bracket 702, and a battery compartment bracket 703. The speaker bracket 701 is clamped and fixed to an inner wall of the inner shell 103. An interior of the speaker bracket 701 is fixedly connected to the speaker 401. The circuit board bracket 702 is integrally formed at a top of the inner shell 103. The circuit board bracket 702 is configured to screw and fix the control circuit board 500. The battery compartment bracket 703 encloses a side wall of the battery compartment 104. The battery compartment bracket 703 is configured to clamp and fix the rechargeable battery 601.

Specifically, the speaker bracket 701 is clamped and fixed to the inner wall of the inner shell 103, and is fixedly connected to the speaker 401, to accurately limit the sound production assembly 400, thus avoiding vibration displacement of the speaker 401 during working and ensuring stable sound effect outputting.

The circuit board bracket 702 is integrally formed at the top of the inner shell 103, and the control circuit board 500 is fixed in a screwing manner, thus preventing an electrical connection failure caused by shaking of the control circuit board 500 and ensuring smooth transmission of a control instruction. The battery compartment bracket 703 encloses a side wall of the battery compartment 104, and clamps and fixes the rechargeable battery 601 to avoid power supplying interruption caused by battery displacement. The three sets of brackets for the internal supporting structure 700 achieve independent positioning and layered fixation of the assemblies, to ensure that the assemblies inside the main housing 100 are arranged neatly without interference. Meanwhile, the cooperation between integrated forming and clamped screwing enhances overall structural strength of the equipment and reduces an assembling difficulty. This structurally eliminates faults caused by movement and looseness of the internal assemblies and provides a solid structural guarantee for stable operations of lighting, sound effect, control, and power supplying functions.

In this embodiment, the decorative ring 120 is made of a metal material or a frosted plastic material, and the decorative ring 120 surrounds and is arranged outside the functional keys 112 of the control panel assembly 110.

Specifically, the decorative ring 120 made of the metal material or the frosted plastic material is embedded to the top of the main housing 100 and surrounds the functional keys 112 of the control panel assembly 110. The metal material can enhance the appearance texture and delicacy of the equipment, while the frosted plastic material has both an anti-slip effect and an aesthetic effect. The surrounding structure of the decorative ring 120 provides edge protection for the functional keys 112, thus avoiding a user from bumping edges of the keys during operation. Meanwhile, the top assemblies are visually distinguished, so that the layout of the functional keys 112 is clearer. It is convenient for a user to quickly identify the positions of the keys, and an overall user experience and appearance of the product are enhanced.

It should be finally noted that: The foregoing embodiments are merely preferred embodiments of the present disclosure, but not intended to limit the present disclosure. Although the present disclosure is described in detail with reference to the foregoing embodiments, a person of ordinary skill in the art may still make modifications to the technical solutions described in the foregoing respective embodiments or make equivalent replacements to partial technical features thereof. Any modification, equivalent replacement, and improvement made within the spirit and scope of the present disclosure shall fall within the protection scope of the present disclosure.

What is claimed is:

1. A sleep-aid audio device, comprising:
a main housing (100), wherein a control panel assembly (110) is mounted at a top of the main housing (100); one side of the main housing (100) is connected to a wristband assembly (200) through a spring pin (210); a wristband buckle (220) is mounted at a top of the wristband assembly (200);
a sound chamber shell (101), a lampshade (102), an inner shell (103), and a battery compartment (104) are sequentially mounted inside the main housing (100) from bottom to top; a lighting assembly (300) is mounted on an inner side of the lampshade (102); a sound production assembly (400) is mounted on an inner side of the sound chamber shell (101); a control circuit board (500) is arranged at a top of the battery compartment (104); a battery assembly (600) is mounted inside the battery compartment (104); an internal supporting structure (700) is further disposed within the main housing (100); the internal supporting structure (700) is fixedly connected to the lighting assembly (300), the sound production assembly (400), the control circuit board (500), and the battery assembly (600) respectively; and
a decorative ring (120) is mounted at the top of the main housing (100) in an embedded manner.

2. The sleep-aid audio device according to claim 1, wherein the control panel assembly (110) comprises a control panel (111); the control panel (111) is mounted at an inner top end of the main housing (100) in an abutting manner; a plurality of functional keys (112) that penetrate through the top of the main housing (100) are mounted on a surface of the control panel (111); the functional keys (112) are electrically connected to the control circuit board (500); the functional keys (112) comprise a power key (1121), a volume increase/decrease key (1122), a mode switching key (1123), a timer key (1124), a lighting key (1125), a child lock key (1126), a Bluetooth key, and a sound effect selection key (1127); at least three indicator lights (113) penetrating through the top of the main housing (100) are further embedded into the surface of the control panel (111); and the indicator lights (113) are electrically connected to the control circuit board (500).

3. The sleep-aid audio device according to claim 2, wherein an inner wall of the lampshade (102) is coated with a light guiding coating (114), and the lampshade (102) is sleeved inside the sound chamber shell (101).

4. The sleep-aid audio device according to claim 1, wherein the wristband assembly (200) comprises a wristband body (201) and the wristband buckle (220); a buckle hole is formed in a surface of the wristband body (201); the wristband body (201) is wound end-to-end and is fixed through the wristband buckle (220) passing through the buckle hole, to form an annular band; one end of the wristband body (201) forming the annular band is internally inserted with the spring pin (210); two ends of the spring pin (210) are inserted into open slots (1031) formed in an outer side of the inner shell (103).

5. The sleep-aid audio device according to claim 1, wherein the lighting assembly (300) comprises a light-emitting diode (LED) board (301); the LED board (301) is of an annular plate-like structure; the LED board (301) is fixed to a top of the lampshade (102) in an abutting manner; a plurality of LEDs (302) are arrayed on a surface of the LED board (301); the lampshade (102) is made of high-transparency frosted acrylic material; and the lampshade (102) is covered outside the LEDs (302).

6. The sleep-aid audio device according to claim 1, wherein the sound production assembly (400) comprises a speaker (401) and a diaphragm (403); the speaker (401) is fixedly mounted in a sound chamber (402) enclosed by the sound chamber shell (101) and the internal supporting structure (700); the sound chamber (402) is of a closed chamber structure; the diaphragm (403) is mounted at a sound production end of the speaker (401) in an abutting manner; and the sound chamber shell (101) is made of a transparent plastic material.

7. The sleep-aid audio device according to claim 1, wherein the control circuit board (500) comprises a main control chip (501) and a storage module (502); the main control chip (501) is electrically connected to the functional keys (112), the lighting assembly (300), the sound production assembly (400), and the battery assembly (600) respectively; the storage module (502) is electrically connected to the main control chip (501); sleep-aid audios are pre-stored in the storage module (502); and the main control chip (501) is also integrated with a Bluetooth chip.

8. The sleep-aid audio device according to claim 1, wherein the battery assembly (600) comprises a rechargeable battery (601) and a charging interface (602); the rechargeable battery (601) is clamped and fixed inside the battery compartment (104); the rechargeable battery (601) is electrically connected to the control circuit board (500); the charging interface (602) is embedded in an outer charging slot of the main housing (100); and the charging interface (602) is electrically connected to the rechargeable battery (601).

9. The sleep-aid audio device according to claim 1, wherein the internal supporting structure (700) comprises a speaker bracket (701), a circuit board bracket (702), and a battery compartment bracket (703); the speaker bracket (701) is clamped and fixed to an inner wall of the inner shell (103); an interior of the speaker bracket (701) is fixedly connected to the speaker (401); the circuit board bracket (702) is integrally formed at a top of the inner shell (103); the circuit board bracket (702) is configured to screw and fix the control circuit board (500); the battery compartment bracket (703) encloses a side wall of the battery compartment (104); and the battery compartment bracket (703) is configured to clamp and fix the rechargeable battery (601).

10. The sleep-aid audio device according to claim 1, wherein the decorative ring (120) is made of a metal material or a frosted plastic material, and the decorative ring (120) surrounds and is arranged outside the functional keys (112) of the control panel assembly (110).
